(19) United States
(12) Patent Application Publication
(10) Pub. No.: US 2021/0141208 A1
(43) Pub. Date: May 13, 2021

(54) ILLUMINATION OPTICAL SYSTEM AND ENDOSCOPE SYSTEM

(71) Applicant: OLYMPUS CORPORATION, Tokyo (JP)

(72) Inventor: Kazunari HANANO, Tokyo (JP)

(73) Assignee: OLYMPUS CORPORATION, Tokyo (JP)

(21) Appl. No.: 17/124,539

(22) Filed: Dec. 17, 2020

Related U.S. Application Data
(63) Continuation of application No. PCT/JP2018/023800, filed on Jun. 22, 2018.

(51) Int. Cl. G02B 23/24; G02B 26/08

(52) U.S. Cl. CPC G02B 23/243; G02B 23/2484; G02B 26/0833; G02B 23/2461

(57) ABSTRACT

An illumination optical system includes a light source, a deflecting element that deflects light from the light source, and a light guide lens group that includes at least one lens and that guides the light deflected by the deflecting element to an input end of a light guide member. The deflecting element changes a deflection angle of the light by deflecting the light input to the deflecting element so as to change an incidence angle of the light entering the input end.

### CROSS-REFERENCE TO RELATED APPLICATIONS

This is a continuation of International Application PCT/JP2018/023800 which is hereby incorporated by reference herein in its entirety.

### TECHNICAL FIELD

The present invention relates to illumination optical systems and endoscope systems.

### BACKGROUND ART

A conventional illumination optical system for an endoscope is equipped with a light source, an optical fiber, and an illumination lens provided at the distal end of a scope (e.g., see Patent Literatures 1 to 5). Light output from the light source is optically guided by the optical fiber and is radiated onto a subject from the illumination lens.

### CITATION LIST

### Patent Literature

{PTL 1}
The Publication of Japanese Patent No. 458843
{PTL 2}
Japanese Unexamined Patent Application, Publication No. 2002-98913
{PTL 3}
Japanese Unexamined Patent Application, Publication No. 2016-2302
{PTL 4}
Japanese Unexamined Patent Application, Publication No. 2010-243874
{PTL 5}
Japanese Unexamined Patent Application, Publication No. 2005-328990

### SUMMARY OF INVENTION

An aspect of the present invention provides an illumination optical system including a light source, a deflecting element that deflects light from the light source, and a light guide lens group that includes at least one lens and that guides the light deflected by the deflecting element to an input end of a light guide member. The deflecting element changes a deflection angle of the light by deflecting the light input to the deflecting element so as to change an incidence angle of the light entering the input end.

Another aspect of the present invention provides an illumination optical system including a light source and a deflecting element that deflects light from the light source toward an input end of a light guide member and that is disposed at a position optically conjugate with the input end. The deflecting element changes a deflection angle of the light by deflecting the light input to the deflecting element so as to change an incidence angle of the light entering the input end.

### BRIEF DESCRIPTION OF DRAWINGS

FIG. 1 illustrates the overall configuration of an endoscope system according to an embodiment of the present invention.

FIG. 2A illustrates the overall configuration of an illumination optical system in the endoscope system in FIG. 1.

FIG. 2B illustrates a state where a deflection angle of illumination light is changed by using a galvanometer mirror in the illumination optical system in FIG. 2A.

FIG. 2C illustrates a state where the deflection angle of the illumination light is further changed by using the galvanometer mirror in the illumination optical system in FIG. 2A.

FIG. 3A illustrates light distribution characteristics of the illumination light output from an output end of a light guide member with respect to the deflection angle of the illumination light shown in FIG. 2A.

FIG. 3B illustrates light distribution characteristics of the illumination light output from the output end of the light guide member with respect to the deflection angle of the illumination light shown in FIG. 2B.

FIG. 3C illustrates light distribution characteristics of the illumination light output from the output end of the light guide member with respect to the deflection angle of the illumination light shown in FIG. 2C.

FIG. 3D illustrates light distribution characteristics of the illumination light when the deflection angle of the illumination light is changed over time by using the galvanometer mirror.

FIG. 4A illustrates the overall configuration of a modification of the illumination optical system in FIG. 2A.

FIG. 4B illustrates a state where the deflection angle of the illumination light is changed by using a MEMS mirror device in the illumination optical system in FIG. 4A.

FIG. 5A illustrates light distribution characteristics of the illumination light output from the output end of the light guide member with respect to the deflection angle of the illumination light shown in FIG. 4A.

FIG. 5B illustrates light distribution characteristics of the illumination light output from the output end of the light guide member with respect to the deflection angle of the illumination light shown in FIG. 4B.

### DESCRIPTION OF EMBODIMENTS

An illumination optical system 1 and an endoscope system 100 according to an embodiment of the present invention will be described below with reference to the drawings.

As shown in FIG. 1, the endoscope system 100 according to this embodiment includes a long scope 2, a light source device 3 connected to the base end of the scope 2, and an image processor 4. The endoscope system 100 also includes an imaging optical system 5 that acquires an image of a subject A, a light guide member 6, the illumination optical system 1 that supplies illumination light L for illuminating the field of view of the imaging optical system 5 to the light guide member 6, and a controller 7 that controls the illumination optical system 1.

The imaging optical system 5 includes an imaging lens 5a and an image sensor 5b. The imaging lens 5a is disposed at the distal-end surface of the scope 2 and forms an image of light from the subject A. The image sensor 5b is disposed within the scope 2 and generates an image signal by acquiring the image of the subject A formed by the imaging lens 5a. The image signal is transmitted from the image sensor 5b to the image processor 4. The image processor 4 generates an image from the image signal and causes a display device (not shown) to display the image.

The light guide member 6 is a long optical member that optically guides the illumination light L and is disposed in the longitudinal direction within the scope 2 from the base end to near the distal end of the scope 2. The light guide member 6 has an input end 6a at the base end and an output end 6b at the distal end. The light guide member 6 optically guides the illumination light L from the input end 6a to the output end 6b and outputs the illumination light L from the output end 6b. At the distal-end surface of the scope 2, an illumination lens 8 is disposed at a position facing the output end 6b. The illumination lens 8 scatters the illumination light L output from the output end 6b and outputs the illumination light L toward the subject A.

For example, as shown in FIG. 2A, such a light guide member 6 is constituted of an optical fiber bundle 61 and a light guide rod 62 connected to the base end of the optical fiber bundle 61. The illumination light L emitted from the light source 11 of the illumination optical system 1 normally has an intensity distribution in which the intensity decreases from the center toward the periphery. The light guide rod 62 has a light scattering function and makes the intensity of the illumination light L uniform.

As shown in FIGS. 2A to 2C, the illumination optical system 1 includes the light source 11 that emits the illumination light L, a galvanometer mirror (deflecting element) 12 that deflects the illumination light L from the light source 11 toward the input end 6a of the light guide member 6, a first lens group (focusing lens group) 13 disposed between the light source 11 and the galvanometer mirror 12, and a second lens group (light guide lens group) 14 disposed between the galvanometer mirror 12 and the input end 6a. The light source 11, the galvanometer mirror 12, the first lens group 13, and the second lens group 14 are disposed within the light source device 3.

The light source 11 is a solid-state light source, such as an LED (light-emitting diode).

The first lens group 13 includes at least one lens. The first lens group 13 forms an image of the light source 11 by focusing the illumination light L from the light source 11.

The second lens group 14 includes at least one lens. The second lens group 14 focuses the illumination light L deflected by the galvanometer mirror 12 onto the end surface of the input end 6a. The galvanometer mirror 12 is disposed at a position optically conjugate with the input end 6a by means of the second lens group 14.

The galvanometer mirror 12 is disposed near the image of the light source 11. The galvanometer mirror 12 is disposed on the optical axis of the illumination light L between the light source 11 and the galvanometer mirror 12 and is rotatable around a rotation axis that is orthogonal to the optical axis. The galvanometer mirror 12 deflects the illumination light L from the light source 11 in a direction parallel to or substantially parallel to the optical axis of the light guide member 6.

As shown in FIGS. 2B and 2C, when the galvanometer mirror 12 is rotated, an incidence angle θ of the illumination light L entering the input end 6a of the light guide member 6 via the second lens group 14 changes. The incidence angle θ is an angle formed between the optical axis of the illumination light L and the optical axis of the light guide member 6. FIG. 2A illustrates a state where the illumination light L is deflected by the galvanometer mirror 12 along the optical axis of the second lens group 14 and the light guide member 6. FIG. 2B illustrates a state where the deflection angle by which the illumination light L is deflected in accordance with rotation of the galvanometer mirror 12 is changed from the deflection angle in FIG. 2A. FIG. 2C illustrates a state where the deflection angle by which the illumination light L is deflected in accordance with further rotation of the galvanometer mirror 12 is further changed from the deflection angle in FIG. 2A.

The illumination light L is optically guided in the longitudinal direction while repeatedly undergoing reflection within the light guide member 6. In the light guide member 6, the illumination light L is optically guided also in the circumferential direction. Furthermore, the angle of the illumination light L relative to the optical axis of the light guide member 6 is maintained. Therefore, as shown in FIGS. 3A to 3C, the illumination light L output from the output end 6b is orbicular, and the light distribution of the illumination light L output from the output end 6b is symmetrical with respect to the center (0°). Furthermore, the output angle of the illumination light L from the output end 6b is equal to the incidence angle θ of the illumination light L entering the input end 6a. Therefore, the light distribution of the illumination light L output from the output end 6b changes in accordance with the incidence angle θ of the illumination light L entering the input end 6a. In light distribution curves in FIGS. 3A to 3C, the optical axis of the light guide member 6 corresponds to 0°.

In detail, the illumination light L emitted from the light source 11 has a light distribution in which the intensity decreases from the center toward the periphery. As shown in FIG. 2A, when the illumination light L enters the input end 6a in a direction parallel to the optical axis of the light guide member 6 (i.e., when the incidence angle θ is) 0°, the light distribution of the illumination light L output from the output end 6b is similar to the light distribution of the illumination light L emitted from the light source 11 in that it is a highly-directional narrow-angle light distribution having a peak intensity at the center, as shown in FIG. 3A.

As shown in FIG. 2B, when the deflection angle of the illumination light L changes from the deflection angle in FIG. 2A as a result of rotation of the galvanometer mirror 12 and the illumination light L enters the input end 6a at an angle relative to the optical axis of the light guide member 6, the illumination light L output from the output end 6b widens, as shown in FIG. 3B, as compared with the light distribution in FIG. 3A. As shown in FIG. 2C, when the incidence angle θ of the illumination light L entering the input end 6a further increases as a result of further rotation of the galvanometer mirror 12, the light distribution of the illumination light L output from the output end 6b further widens, as compared with the light distribution in FIG. 3A, so as to become a wide-angle light distribution having a peak intensity at peripheral sections, as shown in FIG. 3C. Accordingly, as the incidence angle θ increases, the light distribution angle indicating the peak intensity shifts away from 0°, and the light distribution of the illumination light L widens.

The controller 7 controls the rotational angle of the galvanometer mirror 12 based on a user command. The user command is input to the controller 7 by using, for example, an input device (not shown) connected to the controller 7.

Next, the operation of the illumination optical system 1 and the endoscope system 100 having the above-described configuration will be described.

In the endoscope system 100 according to this embodiment, the illumination light L in the form of divergent light emitted from the light source 11 is focused near the galvanometer mirror 12 by the first lens group 13, is deflected by the galvanometer mirror 12, and is guided to the input end 6a of the light guide member 6 by the second lens group 14. The illumination light L entering the light guide member 6 from the input end 6a is output from the output end 6b and is radiated onto the subject A from the illumination lens 8.

The illumination light L reflected at the subject A is received by the imaging lens 5a. An image of the subject A formed by the imaging lens 5a is acquired by the image sensor 5b, and an image signal is transmitted from the image sensor 5b to the image processor 4. Then, the image processor 4 generates the image of the subject A from the image signal, and the image is displayed on the display device.

A user determines whether or not the subject A within the field of view of the imaging optical system 5 is appropriately illuminated with the illumination light L based on the image displayed on the display device. If the image has a dark area where it is difficult to observe the subject A, the user inputs a command for rotating the galvanometer mirror 12 to the controller 7 so as to change the angle of the galvanometer mirror 12 in a direction for increasing the intensity of a portion of the illumination light L corresponding to the dark area. If the image has an excessively bright area where it is difficult to observe the subject A, the user inputs a command for rotating the galvanometer mirror 12 to the controller 7 so as to change the angle of the galvanometer mirror 12 in a direction for decreasing the intensity of a portion of the illumination light L corresponding to the excessively bright area.

For example, when a flat subject A is illuminated with the illumination light L having the light distribution shown in FIG. 3A, the central area of the image is bright, whereas the peripheral area of the image is dark. As shown in FIG. 2B or 2C, the user rotates the galvanometer mirror 12 so as to increase the incidence angle θ of the illumination light L entering the input end 6a. Accordingly, the light distribution of the illumination light L from the output end 6b is changed to a wide-angle light distribution shown in FIG. 3B or 3C, thereby reducing the brightness in the central area of the image and increasing the brightness in the peripheral area.

On the other hand, in a case where the subject A is the inner wall of a narrow cavity, such as the intestine, the front side of the cavity corresponding to the peripheral area of the image is bright, whereas the deep side of the cavity corresponding to the central area of the image is dark. As shown in FIG. 2A, the user rotates the galvanometer mirror 12 so as to reduce the incidence angle θ of the illumination light L entering the input end 6a. Accordingly, the light distribution of the illumination light L from the output end 6b is changed to a highly-directional narrow-angle light distribution shown in FIG. 3A, thereby increasing the brightness at the deep side of the cavity in the image and reducing the brightness at the front side of the cavity.

Accordingly, in this embodiment, the incidence angle θ of the illumination light L entering the input end 6a is changed by using the galvanometer mirror 12, so that the light distribution of the illumination light L illuminating the field of view of the imaging optical system 5 can be dynamically changed during observation of the subject A. This is advantageous in that the subject A can be illuminated appropriately in accordance with the imaging conditions or the imaging target, so that an image with an appropriate degree of contrast can be provided.

Furthermore, the entire illumination light L entering the galvanometer mirror 12 from the light source 11 is deflected by the galvanometer mirror 12, is guided to the input end 6a by the second lens group 14, and is radiated onto the subject A. Accordingly, the illumination light L emitted from the light source 11 is used for illuminating the subject A without loss. This is advantageous in that the illumination efficiency can be improved.

Although it is possible to perform an adjustment to make the dark area in the image brighter by image processing, this method may cause noise to occur in the image or may cause the brightness in the surrounding bright area to become saturated. Moreover, it is also possible to adjust the brightness of the image by using a mechanical aperture stop provided in the illumination optical system 1 or the imaging optical system 5. However, this method may make it difficult to illuminate a distant subject A with sufficient brightness due to loss of light quantity, or may cause heat buildup. In contrast, this embodiment is advantageous in that the light distribution of the illumination light L is adjusted so that the degree of contrast in the image can be adjusted without causing noise to occur in the image or without loss of light quantity.

As an alternative to this embodiment in which the deflecting element is the galvanometer mirror 12, the deflecting element may be a MEMS mirror device 15.

FIGS. 4A and 4B illustrate a configuration example of an illumination optical system 10 that uses the MEMS mirror device 15. The illumination optical system 10 includes the light source 11, the MEMS mirror device 15, a first lens group (collimator lens group) 16 disposed between the light source 11 and the MEMS mirror device 15, and a second lens group (light guide lens group) 17 disposed between the MEMS mirror device 15 and the input end 6a.

The MEMS mirror device 15 has a plurality of micromirrors arranged in a plane. The angle of each micromirror relative to the illumination light L from the light source 11 is adjustable by rotating the micromirror around a rotation axis. Each micromirror deflects the illumination light L from the light source 11 in a direction parallel to or substantially parallel to the optical axis of the light guide member 6. The MEMS mirror device 15 can change the deflection angle of the illumination light L by each micromirror in a continuous or stepwise fashion.

The micromirrors are provided over a wider range than the beam of the illumination light L input from the light source 11 via the first lens group 16. The MEMS mirror device 15 changes all the micromirrors to the same angle. Accordingly, as shown in FIGS. 4A and 4B, the illumination light L entering the MEMS mirror device 15 is entirely deflected toward the input end 6a, and the incidence angle θ of the illumination light L entering the input end 6a of the light guide member 6 via the second lens group 17 changes in accordance with a change in the angle of the micromirrors.

The first lens group 16 includes at least one lens. The first lens group 16 uses the at least one lens to substantially collimate the illumination light L in the form of divergent light emitted from the light source 11, and outputs the substantially collimated light toward the MEMS mirror device 15.

The second lens group 17 includes at least one lens. The second lens group 17 guides the illumination light L, the deflection direction of which is changed by the MEMS mirror device 15, to the input end 6a. In the reference drawings, the second lens group 17 includes a pair of lenses. The lens at the MEMS mirror device 15 side receives the illumination light L deflected by the MEMS mirror device 15, whereas the lens at the light guide member 6 side outputs the illumination light L toward the input end 6a.

FIG. 4A illustrates a state where the illumination light L enters the input end 6a in a direction parallel to the optical axis of the light guide member 6. In this state, the light distribution of the illumination light L output from the output end 6b is a highly-directional narrow-angle light distribution having a peak intensity at the center, as shown in FIG. 5A.

As shown in FIG. 4B, when the deflection angle of the illumination light L changes from the deflection angle in FIG. 4A as a result of rotation of the micromirrors, the incidence angle θ of the illumination light L entering the input end 6a increases. Thus, as shown in FIG. 5B, the light distribution of the illumination light L output from the output end 6b widens, as compared with the light distribution in FIG. 5A.

In this embodiment, the deflecting element 12 or 15 may change the deflection angle of the illumination light L over time between a plurality of angles.

For example, when the galvanometer mirror 12 is repeatedly rotated at high speed among the deflection angle in FIG. 2A, the deflection angle in FIG. 2B, and the deflection angle in FIG. 2C, the light distributions shown in FIGS. 3A, 3B, and 3C are temporally superposed on one another. As a result, the light distribution of the illumination light L output from the output end 6b is the time average of the light distributions in FIGS. 3A, 3B, and 3C, as shown in FIG. 3D, and has high intensity from the center to the periphery.

Accordingly, the deflection angle of the illumination light L is changed over time by using the deflecting element 12 or 15, so that various light distributions constituted of combinations of a plurality of light distributions are realized. Consequently, the light distribution of the illumination light L output from the output end 6b can be controlled to a desired light distribution. The deflecting element 12 or 15 may change the deflection angle in a stepwise fashion among the three angles in FIGS. 2A, 2B, and 2C, or in a continuous fashion between the two angles in FIGS. 2A and 2C. The deflecting element 12 or 15 may change the deflecting angle among four or more angles.

In this embodiment, the controller 7 may change the quantity of light emitted from the light source 11 in accordance with the deflection angle of the illumination light L deflected by the deflecting element 12 or 15.

As the light distribution of the illumination light L changes, the brightness in each of the central area and the peripheral area of the illumination light L changes. For example, as a result of changing the light distribution of the illumination light L from the light distribution in FIG. 5A to the light distribution in FIG. 5B, the brightness of the central area in the image decreases. In such a case, the controller 7 may increase the quantity of light emitted from the light source 11. Alternatively, as a result of changing the light distribution of the illumination light L from the light distribution in FIG. 5B to the light distribution in FIG. 5A, the brightness of the central area in the image increases. In such a case, the controller 7 may reduce the quantity of light emitted from the light source 11.

In this embodiment, the controller 7 may control at least one of the deflecting element 12 or 15 and the light source 11 based on the image of the subject A.

For example, the controller 7 detects the brightness of the central area and the brightness of the peripheral area in the image based on pixel values. If the peripheral area is darker than the central area, the controller 7 changes the deflection angle of the illumination light L deflected by the deflecting element 12 or 15 in the direction for widening the light distribution of the illumination light L output from the output end 6b, thereby increasing the brightness of the peripheral area. If the central area is darker than the peripheral area, the controller 7 changes the deflection angle of the illumination light L deflected by the deflecting element 12 or 15 in the direction for narrowing the light distribution of the illumination light L output from the output end 6b, thereby increasing the brightness of the central area.

As an alternative to or in addition to changing the deflection angle of the illumination light L deflected by the deflecting element 12 or 15, the controller 7 may change the quantity of light emitted from the light source 11 in accordance with the brightness of the image.

The above-described embodiment leads to the following aspects.

An aspect of the present invention provides an illumination optical system including a light source, a deflecting element that deflects light from the light source, and a light guide lens group that includes at least one lens and that guides the light deflected by the deflecting element to an input end of a light guide member. The deflecting element changes a deflection angle of the light by deflecting the light input to the deflecting element so as to change an incidence angle of the light entering the input end.

According to this aspect, the light emitted from the light source is deflected by the deflecting element, is guided to the input end of the light guide member by the light guide lens group, and is output from an output end of the light guide member, thereby illuminating a subject. The light distribution of the light output from the output end of the light guide member is dependent on the incidence angle of the light entering the input end. Therefore, by using the deflecting element to change the incidence angle of the light entering the input end in accordance with the imaging conditions or the imaging target, the light distribution of the illumination light illuminating the subject can be controlled, thereby providing an image with an appropriate degree of contrast.

Another aspect of the present invention provides an illumination optical system including a light source and a deflecting element that deflects light from the light source toward an input end of a light guide member and that is disposed at a position optically conjugate with the input end. The deflecting element changes a deflection angle of the light by deflecting the light input to the deflecting element so as to change an incidence angle of the light entering the input end.

According to this aspect, the light emitted from the light source is deflected by the deflecting element, is input to the input end of the light guide member that is disposed at the position optically conjugate with the deflecting element, and is output from an output end of the light guide member, thereby illuminating a subject. The light distribution of the light output from the output end of the light guide member is dependent on the incidence angle of the light entering the input end. Therefore, by using the deflecting element to change the incidence angle of the light entering the input end in accordance with the imaging conditions or the imaging target, the light distribution of the illumination light illuminating the subject can be controlled, thereby providing an image with an appropriate degree of contrast.

In the above aspect, the deflecting element may change the deflection angle over time. By changing the deflection angle of the light deflected by the deflecting element over time, the light distribution of the light output from the output end of the light guide member changes over time. Specifically, the light distribution of the light illuminating the subject is the time average of a plurality of light distributions. Therefore, illumination light with various light distributions can be realized in accordance with combinations of a plurality of light distributions.

In the above aspect, the deflecting element may be capable of changing the deflection angle among three or more angles.

According to this configuration, the light distribution of the illumination light that illuminates the subject can be adjusted to a more appropriate light distribution.

In the above aspect, the deflecting element may be capable of changing the deflection angle in a continuous fashion.

According to this configuration, the light distribution of the illumination light that illuminates the subject can be changed in a continuous fashion, so as to be adjusted to a more appropriate light distribution.

In the above aspect, the illumination optical system may further include a focusing lens group that includes at least one lens and that is disposed between the light source and the deflecting element. The focusing lens group may form an image of the light source by focusing the light from the light source. The deflecting element may be disposed near the image of the light source and may deflect the light focused by the focusing lens group.

According to this configuration, the light deflected by the deflecting element becomes divergent light, so that divergent light or convergent light enters the input end of the light guide member. Accordingly, a wider light distribution can be realized, as compared with a case where collimated light enters the input end of the light guide member.

In the above aspect, the deflecting element may be a galvanometer mirror disposed on an optical axis of the light from the light source.

By rotating the galvanometer mirror, the deflection angle of the light can be changed, so that the incidence angle of the light entering the end surface of the input end can be changed.

In the above aspect, the illumination optical system may further include a collimator lens group that includes at least one lens and that is disposed between the light source and the deflecting element. The collimator lens group may substantially collimate the light from the light source.

According to this configuration, substantially collimated light enters the input end of the light guide member from the collimator lens group. Accordingly, a narrower-angle highly-directional light distribution can be realized, as compared with a case where divergent light or convergent light enters the input end of the light guide member.

In the above aspect, the deflecting element may be a MEMS (microelectromechanical system) mirror device that includes a plurality of micromirrors and in which an angle of each of the plurality of micromirrors is adjustable.

By changing the angle of each micromirror, the deflection angle of the light can be changed, so that the incidence angle of the light entering the input end can be changed.

In the above aspect, the light source may change a quantity of light emitted therefrom in accordance with the deflection angle of the light deflected by the deflecting element.

As the light distribution of the light output from the output end changes, the brightness of the light illuminating each area of the subject changes. By changing the quantity of light emitted from the light source in accordance with the deflection angle, the subject can be illuminated with appropriate brightness.

Another aspect of the present invention provides an endoscope system including: a light guide member that includes an input end and an output end, that optically guides light entering the input end, and that outputs the light from the output end; the aforementioned illumination optical system; an imaging optical system that acquires an image of a subject illuminated with the light output from the output end of the illumination optical system; and a controller that controls at least one of the deflecting element and the light source. The controller controls at least one of a deflection angle of the light deflected by the deflecting element and a quantity of light emitted from the light source based on the image of the subject acquired by the imaging optical system.

According to this aspect, the subject is illuminated with the light output from the output end of the light guide member of the illumination optical system, and the image of the illuminated subject is acquired by the imaging optical system. The image has bright and dark areas occurring therein in accordance with an irregular shape of the subject or the light distribution of the light from the output end. By controlling at least one of the deflection angle of the light deflected by the deflecting element and the quantity of light emitted from the light source based on the image, the controller can adjust at least one of the light distribution and the quantity of the light output from the output end so that the image has an appropriate degree of contrast.

### REFERENCE SIGNS LIST

1, 10 illumination optical system
2 scope
3 light source device
4 image processor
5 imaging optical system
6 light guide member
6a input end
6b output end
7 controller
8 illumination lens
11 light source
12 galvanometer mirror (deflecting element)
13 first lens group (focusing lens group)
14 second lens group (light guide lens group)
15 MEMS mirror device (deflecting element)
16 first lens group (collimator lens group)
17 second lens group (light guide lens group)
100 endoscope system

1. An illumination optical system comprising:
a light source;
a deflecting element that deflects light from the light source; and
a light guide lens group that comprises at least one lens and that guides the light deflected by the deflecting element to an input end of a light guide member,
wherein the deflecting element changes a deflection angle of the light by deflecting the light input to the deflecting element so as to change an incidence angle of the light entering the input end.

2. An illumination optical system comprising:
a light source; and
a deflecting element that deflects light from the light source toward an input end of a light guide member and that is disposed at a position optically conjugate with the input end,
wherein the deflecting element changes a deflection angle of the light by deflecting the light input to the deflecting element so as to change an incidence angle of the light entering the input end.

3. The illumination optical system according to claim 1,
wherein the deflecting element changes the deflection angle over time.

4. The illumination optical system according to claim 2, wherein the deflecting element changes the deflection angle over time.

5. The illumination optical system according to claim 1,
wherein the deflecting element is capable of changing the deflection angle among three or more angles.

6. The illumination optical system according to claim 2,
wherein the deflecting element is capable of changing the deflection angle among three or more angles.

7. The illumination optical system according to claim 1,
wherein the deflecting element is capable of changing the deflection angle in a continuous fashion.

8. The illumination optical system according to claim 2,
wherein the deflecting element is capable of changing the deflection angle in a continuous fashion.

9. The illumination optical system according to claim 1, further comprising:
a focusing lens group that comprises at least one lens and that is disposed between the light source and the deflecting element,
wherein the focusing lens group forms an image of the light source by focusing the light from the light source, and
wherein the deflecting element is disposed near the image of the light source and deflects the light focused by the focusing lens group.

10. The illumination optical system according to claim 2, further comprising:
a focusing lens group that comprises at least one lens and that is disposed between the light source and the deflecting element,
wherein the focusing lens group forms an image of the light source by focusing the light from the light source, and
wherein the deflecting element is disposed near the image of the light source and deflects the light focused by the focusing lens group.

11. The illumination optical system according to claim 9,
wherein the deflecting element is a galvanometer mirror disposed on an optical axis of the light from the light source.

12. The illumination optical system according to claim 10,
wherein the deflecting element is a galvanometer mirror disposed on an optical axis of the light from the light source.

13. The illumination optical system according to claim 1, further comprising:
a collimator lens group that comprises at least one lens and that is disposed between the light source and the deflecting element,
wherein the collimator lens group substantially collimates the light from the light source.

14. The illumination optical system according to claim 2, further comprising:
a collimator lens group that comprises at least one lens and that is disposed between the light source and the deflecting element,
wherein the collimator lens group substantially collimates the light from the light source.

15. The illumination optical system according to claim 13,
wherein the deflecting element is a MEMS mirror device that comprises a plurality of micromirrors and in which an angle of each of the plurality of micromirrors is adjustable.

16. The illumination optical system according to claim 14,
wherein the deflecting element is a MEMS mirror device that comprises a plurality of micromirrors and in which an angle of each of the plurality of micromirrors is adjustable.

17. The illumination optical system according to claim 1,
wherein the light source changes a quantity of light emitted therefrom in accordance with the deflection angle of the light deflected by the deflecting element.

18. The illumination optical system according to claim 2,
wherein the light source changes a quantity of light emitted therefrom in accordance with the deflection angle of the light deflected by the deflecting element.

19. An endoscope system comprising:
a light guide member that comprises an input end and an output end, that optically guides light entering the input end, and that outputs the light from the output end;
the illumination optical system according to claim 1;
an imaging optical system that comprises an image sensor and that is configured to acquire an image of a subject illuminated with the light output from the output end of the illumination optical system; and
a controller configured to control at least one of the deflecting element and the light source,
wherein the controller is configured to control at least one of a deflection angle of the light deflected by the deflecting element and a quantity of light emitted from the light source based on the image of the subject acquired by the imaging optical system.

20. An endoscope system comprising:
a light guide member that comprises an input end and an output end, that optically guides light entering the input end, and that outputs the light from the output end;
the illumination optical system according to claim 2;
an imaging optical system that comprises an image sensor and that is configured to acquire an image of a subject illuminated with the light output from the output end of the illumination optical system; and
a controller configured to control at least one of the deflecting element and the light source,
wherein the controller is configured to control at least one of a deflection angle of the light deflected by the deflecting element and a quantity of light emitted from the light source based on the image of the subject acquired by the imaging optical system.
